(19) United States
(12) Plant Patent Application Publication
(10) Pub. No.: US 2017/0196147 P1
(43) Pub. Date: Jul. 6, 2017

(54) Phalaenopsis orchid plant named 'PHALODMEL'

(50) ;

(71) Applicant: ANTHURA B.V., Bleiswijk (NL)

(72) Inventor: Martinus Nicolaas Gerardus Van Swieten, Utrecht (NL)

(21) Appl. No.: 14/998,453

(22) Filed: Jan. 5, 2016

(51) Int. Cl. A01H 5/02

(52) U.S. Cl. PLT/311

(57) ABSTRACT

A new and distinct variety of Phalaenopsis plant named ‘PHALODMEL’, particularly characterized by having purple flowers with dark purple lip, 1 to 3 peduncles, an inflorescence that is short to medium long and moderate, leaves that are lanceolate, and is propagated by meristem tissue culture, is disclosed.

Genus and species: Phalaenopsis hybrid.

Variety denomination: ‘PHALODMEL’.

### BACKGROUND OF THE NEW PLANT

The present invention comprises a new and distinct cultivar of Phalaenopsis plant, botanically known as Phalaenopsis of the Orchidaceae family, and hereinafter referred to by the cultivar name ‘PHALODMEL’.

Phalaenopsis comprises a genus of about 60 species of herbaceous perennials many of which, or the hybrids thereof, are suitable for cultivar in the home or greenhouse. Phalaenopsis is predominantly epiphytic or rock dwelling, and is native to tropical Asia, the Malay Archipelago, and Oceania. The species typically has 2-ranked, fleshy, oblong or elliptic leaves affixed to a short central stem (monopodial growth), which vary in size from 12 to 20 cm to over 60 cm. The leaves may be entirely green or mottled with silver grey.

Phalaenopsis orchids, often referred to as ‘Moth Orchids’ in the horticultural trade, are frequently used to furnish cut flowers for the florist trade or sold as flowering potted-plants for home or interiorscape.

Phalaenopsis produces upright or pendent lateral racemes, often with many showy flowers which open in succession beginning with the lowermost. The flowers possess three sepals and three petals; the lateral ones being alike. The lowermost petals, called the labellum, are three-lobed and are often more brightly-colored than the other flower segments. Flower colors include various shades of pink, white, yellow, and red-brown.

Phalaenopsis orchids are typically propagated from seeds. Asexual propagation of Phalaenopsis is often done from off-shoots which arise from the lower bracts of the inflorescence. The resulting plants are detached from the mother plants and may be planted in a suitable substrate.

The new Phalaenopsis ‘PHALODMEL’ is particularly characterized by its attractive and unique purple flowers with dark purple lip, economical propagation by tissue culture, rapid growth, and a plant dimension suitable for packaging and shipping to the market.

‘PHALODMEL’ is a product of a planned breeding program conducted by the inventor in Bleiswijk, The Netherlands.

The new Phalaenopsis ‘PHALODMEL’ originated from a cross made in August 2013 in Bleiswijk, The Netherlands. The female parent is a Phalaenopsis pot plant named ‘22044-0002’ (unpatented), while the male parent is a Phalaenopsis pot plant named ‘21981-0001’ (unpatented). A single plant was selected in February 2007 and has been asexually reproduced repeatedly by meristem tissue culture in Bleiswijk, The Netherlands over a 5.5-year period. The new variety has been found to retain its distinctive characteristics through successive asexual propagations.

Asexual reproduction of ‘PHALODMEL’ by tissue culture was first performed in February 2010 in Bleiswijk, The Netherlands and has demonstrated that the new cultivar is firmly fixed and retained through successive generations of asexual reproduction.

Plant Breeder's Rights for this variety have not been applied for. ‘PHALODMEL’ has not been made publicly available or sold anywhere in the world more than one year prior to the filing date of this application.

### SUMMARY OF THE INVENTION

The following are the most outstanding and distinguishing characteristics of this new cultivar when grown under normal horticultural practices in Bleiswijk, The Netherlands.
1) Purple flowers with dark purple lip;
2) 1 to 3 peduncles;
3) Inflorescence is short to medium long and moderate;
4) Shape of the leaf is lanceolate; and
5) Plants are propagated by meristem tissue culture.

### DESCRIPTION OF THE PHOTOGRAPHS

This new Phalaenopsis plant is illustrated by the accompanying photographs which show the overall plant habit including blooms and foliage of the plant; the colors shown are as true as can be reasonably obtained by conventional photographic procedures. The photographs are of a 50-week old plant grown in a greenhouse in Bleiswijk, The Netherlands in August 2015.

FIG. 1 shows the overall plant habit, including blooms and foliage of ‘PHALODMEL’.

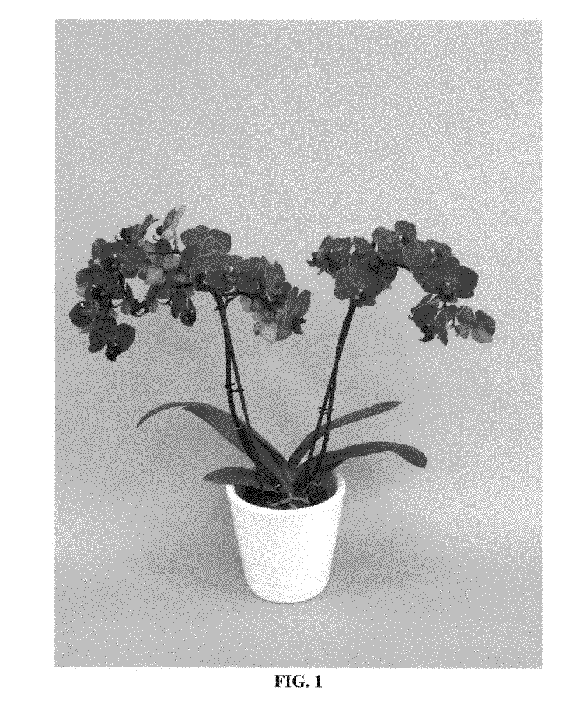

FIG. 2 shows a close-up of a flower of ‘PHALODMEL’.

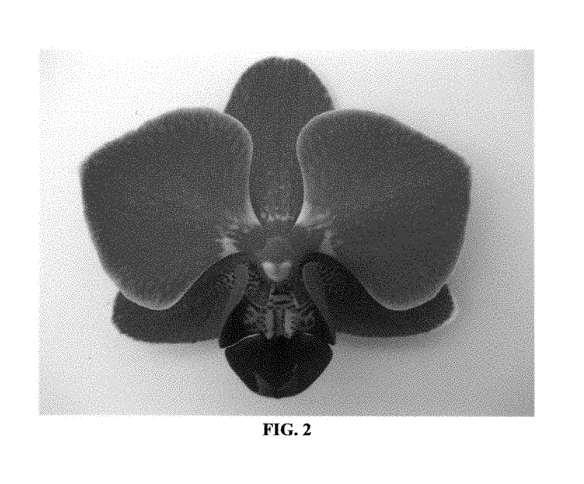

FIG. 3 shows a close-up of the leaves of ‘PHALODMEL’.

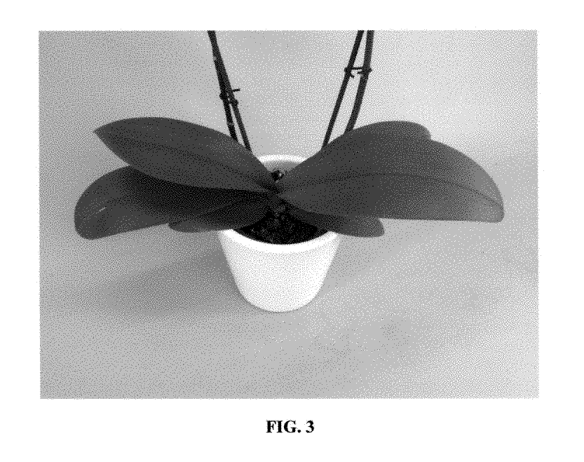

### DESCRIPTION OF THE NEW VARIETY

The following detailed description sets forth the distinctive characteristics of ‘PHALODMEL’. The data which define these characteristics were collected from asexual reproductions carried out in Bleiswijk, The Netherlands. The plant history was taken on 50-week old plants which were planted from a nursery tray in 12 centimeter pots and grown in a greenhouse between 27° C. to 29° C. for 30 weeks, continued by a cooling period of 8 weeks between 18° C. to 20° C. and 12 weeks in a greenhouse of 21° C. Observations were made in August 2015. Color readings were taken under 4-6000 lux natural light in the greenhouse. Color references are primarily to The R.H.S. Colour Chart of The Royal Horticultural Society of London (R.H.S.) (1995).

### DETAILED BOTANICAL DESCRIPTION

Classification:
   Family.—Orchidaceae.
   Botanical.—Phalaenopsis hybrid.
   Common name.—Phalaenopsis.
   Variety name.—‘PHALODMEL’.
Parentage:
   Female parent.—Phalaenopsis cultivar ‘22044-0002’ (unpatented).
   Male parent.—Phalaenopsis cultivar ‘21981-0001’ (unpatented).
Propagation:
   Type.—Meristem tissue culture.
Plant:
   Crop time (time to produce a finished flowering plant).—48 to 50 weeks for a 12 cm pot.
   Growth habit of inflorescence.—Standard, green leaves, panicle.
   Height (including pot, including inflorescence).—43.0 cm to 53.0 cm.
   Width (measured from leaf tips).—30.0 cm to 35.0 cm.
   Vigor.—Strong.
Roots:
   Root description.—Grey-green colored roots with branching lateral roots having light green-purple colored root tips.
Leaves:
   Mature leaves.—Quantity per plant: 5 to 8 leaves are produced before flowering Length (fully expanded): 17.0 cm to 20.0 cm Width: 7.5 cm to 8.5 cm Shape: Lanceolate Base shape: Moderately elongated Apex: Unequal obtuse Leaf blade angle with the petiole (measured from the horizontal position): Between 20 degrees and 40 degrees Leaf margin: Entire Color: Upper surface: RHS 146A Lower surface: RHS 146B Texture: Rough Thickness: 2.6 mm to 3.1 mm Venation: Pattern: Parallel Color of the midvein: Upper surface: RHS 146A Lower surface: RHS 146B.
Peduncle:
   Quantity per plant.—1 to 3.
   Number of flowers per peduncle.—24 to 32.
   Length.—33.0 cm to 38.0 cm.
   Diameter.—6.0 mm to 6.3 mm.
   Strength.—Strong.
   Aspect.—Upright.
   Texture.—Smooth.
   Color.—Mix of green (RHS 146B) and brown (RHS 200A).
   Internode length.—28.0 mm to 38.0 mm.
   Callosities.—None.
Inflorescence description:
   Appearance.—Upright to slightly pendant, panicle inflorescence with bilaterally symmetrical flowers that open in succession beginning with the lowermost flower.
   Inflorescence size.—Height (from base to tip): 190.0 mm to 220.0 mm.
   Flowering time.—First flowers can be expected 10 to 11 months after planting in a 12 cm pot.
   Flower.—Height: 46.0 mm to 51.0 mm Diameter: 55.0 mm to 60.0 mm Depth of lip: 17.0 mm to 19.0 mm.
   Flower longevity.—On the plant: 9 to 11 weeks.
   Fragrance.—Absent.
   Flower bud.—Average size: Medium Length: 15.0 mm to 17.0 mm Width: 13.0 mm to 15.0 mm Shape: Egg shaped Color: Mix of green (RHS 146D) and purple (RHS 77A and 79B).
   Petals.—Arrangement: Open/free Shape: Semi-circular Apex: Obtuse asymmetric Margin: Entire to slightly undulate Length (from base to tip): 29.0 mm to 31.0 mm Width: 30.0 mm to 32.0 mm Color (when fully opened): Upper surface: Basic color: Purple (RHS 78A) with light purple at the base (RHS 78C) Over color: Absent Lower surface: Basic color: Purple (RHS 80B) Over color: Light purple towards the base (RHS 80D).
   Dorsal sepal.—Shape: Elliptic Apex: Rounded symmetric Margin: Entire Length (from base to tip): 28.0 mm to 30.0 mm Width: 20.0 mm to 22.0 mm Color (when fully opened): Upper surface: Basic color: Purple (RHS 78A) with a touch of light purple at the base (RHS 78D) Over color: Absent Lower surface: Basic color: Purple (RHS 80D) Over color: Light purple in the middle (RHS 76C).
   Lateral sepals.—Shape: Ovate Apex: Obtuse asymmetric Margin: Entire Length (from base to tip): 29.0 mm to 31.0 mm Width: 19.0 mm to 21.0 mm Color (when fully opened): Upper surface: Basic color: Purple (RHS 78A) dotted toward the base (RHS 59A) with a light edge (RHS 76C to 76D) Over color: Light green towards the base (RHS 145D) Lower surface: Basic color: Purple (RHS 78B) Over color: Light purple in the middle (RHS 76C) with a touch of greyed-green at the base (RHS 145B).
   Labellum (lip).—Margin: Entire Whiskers: Present Length of whiskers: 3.0 mm to 5.0 mm Color of whiskers: Dark red-purple (RHS 59A and 71A) Pubescence on the lip: Absent.
   Lateral lobe.—Shape: Type V (as described in the International Union for the Protection of New Varieties of Plants (UPOV) Test Guidelines for Phalaenopsis); spatulate. Length (from base to tip): 14.0 mm to 16.0 mm. Width: 10.0 mm to 12.0 mm Color: Dark purple (RHS 59A; 71A and 187A) with a touch of yellow at the base (RHS 6A).
   Apical lobe.—Shape: Trullate Length (from base to tip): 18.0 mm to 20.0 mm Width: 16.0 mm to 18.0 mm Color: Dark purple (RHS 59A; 71A and 187A).
   Callus.—Average size: Small to medium Height: 0.4 cm to 0.5 cm Length: 0.5 cm to 0.6 cm Width: 0.4 cm to 0.5 cm Color: Yellow (RHS 5A) dotted (RHS 71A).
Reproductive organs:
   Arrangement.—The stamens, style and stigmas are fused into a single, short structure called the column, possessing one terminal anther with pollen grains united into pollinia, which are covered by an anther cap. The stigma is located under the column behind the pollinia. The ovary is inferior with three carpels present.
   Column.—Length: 8.0 mm to 10.0 mm Diameter: 4.3 mm to 4.6 mm Color: Purple (RHS 78A).
   Pollinia.—Quantity: 2 Size: 0.8 mm to 1.0 mm Color: Orange (RHS 26A).
   Ovary.—Part of the pedicel with small ribs towards the column Length: 5.0 mm to 7.0 mm Diameter: 1.8 mm to 2.0 mm.
   Pedicel.—Length: 22.0 mm to 25.0 mm Diameter: 2.1 mm to 2.4 mm Color: From inflorescence light greyed-green (RHS 146B) and light purple towards the flower (RHS 78C to 78D).
Disease, pest, and stress resistance: No specific resistance or susceptibility observed
Temperature tolerance: Tolerant to a low temperature of 15° C. and a high temperature of about 30° C.

### COMPARISON WITH PARENTAL AND COMMERCIAL VARIETIES

The female parent ‘22044-0002’ (unpatented) of ‘PHALODMEL’ is no longer available, so a comparison cannot be made.

The male parent ‘21981-0001’ (unpatented) of ‘PHALODMEL’ is no longer available, so a comparison cannot be made.

‘PHALODMEL’ differs from commercial variety ‘PHALDIMXAP’ (U.S. Plant Pat. No. 25,294) in that ‘PHALODMEL’ has a callus that is light yellow dotted, whereas ‘PHALDIMXAP’ has a callus that is darker yellow-orange dotted. Additionally, ‘PHALODMEL’ has flowers with a smaller center than ‘PHALDIMXAP’.

I claim:

1. A new and distinct variety of Phalaenopsis plant named ‘PHALODMEL’, substantially as described and illustrated herein.
